(19) United States
(12) Patent Application Publication
(10) Pub. No.: US 2021/0045712 A1
(43) Pub. Date: Feb. 18, 2021

(54) HOLSTER FOR ULTRASOUND IMAGING DEVICE

(71) Applicant: Butterfly Network, Inc., Guilford, CT (US)

(72) Inventors: Matthew de Jonge, Brooklyn, NY (US); Neil Ferrier, Greenville, SC (US); Kevin Coss, Irvine, CA (US)

(73) Assignee: Butterfly Network, Inc., Guilford, CT (US)

(21) Appl. No.: 16/991,217

(22) Filed: Aug. 12, 2020

Related U.S. Application Data
(60) Provisional application No. 62/886,945, filed on Aug. 14, 2019.

(51) Int. Cl. A61B 8/00

(52) U.S. Cl. CPC A61B 8/4227; A61B 8/4218

(57) ABSTRACT

Described herein is an apparatus and method for attaching an ultrasound imaging device to a user. The apparatus includes a holster with a cradle for receiving at least a portion of the imaging device and a strap attachable to the cradle to secure the probe in the holster. In some embodiments, the strap extends diagonally across a portion of the imaging device when the imaging device is the holster. The strap may be formed of a flexible material while the cradle is formed of a rigid material. A width of the strap may decrease in an outwardly direction, from a first end of the strap to a second end of the strap.

### FIELD

Generally, the aspects of the technology described herein relate to holsters to hold an item on a user. Some aspects relate to a holster for an ultrasound imaging device.

### DISCUSSION OF THE RELATED ART

Ultrasound devices may be used to perform diagnostic imaging and/or treatment, using sound waves with frequencies that are higher than those audible to humans. Ultrasound imaging may be used to see internal soft tissue body structures. When pulses of ultrasound are transmitted into tissue (e.g., by using an ultrasound imaging device), sound waves are reflected off the tissue, with different tissues reflecting varying degrees of sound. These reflected sound waves may then be recorded and displayed as an ultrasound image to the operator.

When not actively using an ultrasound imaging device, an operator may wish to temporarily hold the device to his or her body without having to actively hold the device with his or her hand.

### SUMMARY

According to one embodiment, a holster for holding an ultrasound imaging device on a user is disclosed. The holster includes a cradle arranged to receive at least a portion of the ultrasound imaging device, and a strap having first and second ends, wherein the first end of the strap is attached to the holster and the second end of the strap is removably attachable to the cradle to secure the ultrasound imaging device in the cradle.

According to another embodiment, a method includes inserting an ultrasound imaging device into a cradle of a holster, stretching a strap over at least a portion of the ultrasound imaging device, and securing the strap to the cradle.

According to another embodiment, a holster for holding an ultrasound imaging device on a user is disclosed. The holster includes a cradle arranged to receive at least a portion of the ultrasound imaging device, the cradle having first and second arms arranged to wrap around at least a portion of the ultrasound imaging device, and a strap having first and second ends, the first end of the strap being attached to the holster. The first and second arms are arranged to deflect outwardly away from one another when the ultrasound imaging device is inserted into the cradle.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 shows a holster for holding an ultrasound imaging device according to embodiments disclosed herein;

FIG. 2 is the holster of FIG. 1, with a strap of the holster attached to a cradle of the holster;

FIG. 3 is a left side view of the holster of FIG. 1, with a clip of the holster shown removed;

FIG. 4 is a rear view of the holster of FIG. 3;

FIG. 5 is a front view of the holster of FIG. 3;

FIG. 6 is a top view of the holster of FIG. 3;

FIG. 7 illustrates an ultrasound imaging device being inserted into a cradle of the holster of FIG. 1;

FIG. 8 shows an ultrasound imaging device seated in a cradle of a holster; and

FIG. 9 shows the holster of FIG. 8, with a strap attached to the cradle and the ultrasound imaging device secured in the holster.

### DETAILED DESCRIPTION

Conventional ultrasound systems are large, complex, and expensive systems that are typically only purchased by large medical facilities with significant financial resources. For example, ultrasound systems are typically arranged on a large cart that is setup in an exam room or is wheeled around to a patient's bedside for testing. As is known, the carts typically include a screen for viewing and transducers that are stored in the cart.

Recently, cheaper, portable, and less complex ultrasound imaging devices have been introduced. Such imaging devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application) and published as U.S. Pat. Pub. No. 2017/0360397 A1, which is incorporated by reference herein in its entirety. The reduced cost and increased portability of these new ultrasound devices may make them significantly more accessible to the general public than conventional ultrasound devices. For example, in some embodiments, the ultrasound imaging device may be connected to a mobile electronic device, such as a smart phone, which may act as the display for a user performing diagnostic imaging and/or treatment on a patient with the ultrasonic imaging device.

The inventors have recognized that advantages may be realized if the user is able to hold the ultrasound imaging device, also referred to herein as an ultrasound probe or probe, on his or her person at all desired times. For example, the user may be able to keep the ultrasound probe with him or her while visiting patients during rounds such that imaging and/or treatment may be performed when needed, without the user having to first reserve, locate, and/or transport an ultrasound cart to the patient. The inventors have also recognized that advantages may be realized if the user is able to removably attach the ultrasound probe to his or her clothing, or to another personal item, such as to a bag. For example, the user may clip the ultrasound probe onto the waist area of the user's pants or skirt, to a pocket, or to a jacket. The inventors further recognized that the ultrasound imaging device would benefit by being removably attachable to a holder, also referred to herein as a holster, that, in turn, may attach the ultrasound probe to the user's clothes or other personal item. For example, the user may remove the ultrasound probe from a holster attached to his or her jacket for use, and thereafter return the ultrasound probe to the holster once imaging and/or treatment is performed. Examples of holsters used to hold an ultrasound probe are shown and described in the U.S. patent application Ser. No. 16/172,618, titled “HOLSTER FOR ULTRASOUND IMAGING DEVICE” (assigned to the assignee of the instant application) and published as U.S. Pat. App. Pub. No. 2019/0125064, which is incorporated by reference herein in its entirety.

The inventors have further recognized that existing holsters do not always provide a satisfactory solution in all aspects. For example, the inventors have discovered that for some holsters, even though the probe may be secured in the holster, the cord may not remain contained by the holster. As an illustrative example, such as where the user wraps at least a portion of the cord around a part of the probe, such as around the neck of the probe, the cord may fall off and/or unravel from the probe while the probe is secured in the holster. This may be inconvenient as the user may need to repeatedly remove and reattach the probe to the holster to secure the cord. It also may lead to a damaged cord or even a damaged probe, such as if a loose cord were to snag on something and pull the probe out of the holster or pull the holster with an attached probe off of the user. In view of the above, the inventors have recognized that the ultrasound probe would benefit from having a holster with a cable management system which permits the cord to remain on and/or around the probe when the probe is secured in the holster.

The inventors have also appreciated that for some holsters, the probe may move around in the holster even though the probe is secured in the holster. For example, a probe may not remain vertically aligned in the holster when the probe is seated in the holster. The inventors have recognized that it would be beneficial to have a holster which maintains the position of the probe relative to the holster when the probe is secured in the holster.

The inventors have further appreciated that the size and arrangement of some holsters may make the holsters uncomfortable or even obstructive for the user to wear when the probe is being used. The inventors have recognized that the ultrasound probe may benefit from a holster that has a smaller profile when the probe is in use. For example, advantages may be realized if the holster is at least partially collapsible when the ultrasound is not in the holster. Advantages also may be realized by having a holster that may be at least partially removable from a user's clothing or other item while maintaining the probe in the holster. For example, the clip used to attach the holster to the user's clothing (such as a belt) may be removably attachable such that the user may keep the clip attached to the user's clothing or other personal item while removing the remainder of the holster (with or without the probe attached). Advantages also may be realized by having a holster with a quick release option available when the user needs to access the probe for testing.

Embodiments disclosed herein include a holster configured to hold the ultrasound imaging device, or ultrasound probe, on a user. For example, the holster may be configured to retain the ultrasound probe in the holster and also to attach the holster to a portion of the user's clothing or other personal item. In some embodiments, the user may clip the holster to the waist area of a pair of pants or a skirt, or to a pocket on the clothes of the user, and insert and secure the probe in the holster. In such embodiments, the user may to carry the ultrasound probe on himself or herself, from place to place (e.g., between patient's rooms), and remove the ultrasound probe from the holster when the user is ready to perform imaging and/or treatment. As will be appreciated, once the imaging and/or treatment is complete, the user may return the probe to the holster and secure the probe into the holster.

In some embodiments, the holster includes include one or more retainers to hold the probe relative to the holster. For example, the probe may have one or more retainers to hold the probe in the holster and/or to secure the probe in the holster. As will be described, in some embodiments, the one or more retainers also may serve to help secure the cord on and/or around the probe. In some embodiments, the one or more retainers may be arranged to maintain the position of the probe relative to the holster.

In some embodiments, as will be described, the retainers may include a cradle and a strap. In some embodiments, the cradle is arranged to wrap around at least a portion of the probe, such as at least a portion of a central region of the probe. For example, the cradle may extend around a rear of the probe, toward and around at least a portion of a front of the probe. The cradle also may extend all the way around the portion of the probe in some embodiments.

In some embodiments, the cradle may be arranged to hold the probe in an over-center arrangement. For example, the cradle may be flexibly resilient and resist insertion of the probe into the cradle. But, once the widest point of the probe body passes an upper edge of the cradle while being inserted, the cradle no longer resist the insertion and instead starts to push the probe towards its cradled position, or at least no longer resists the cradling of the probe.

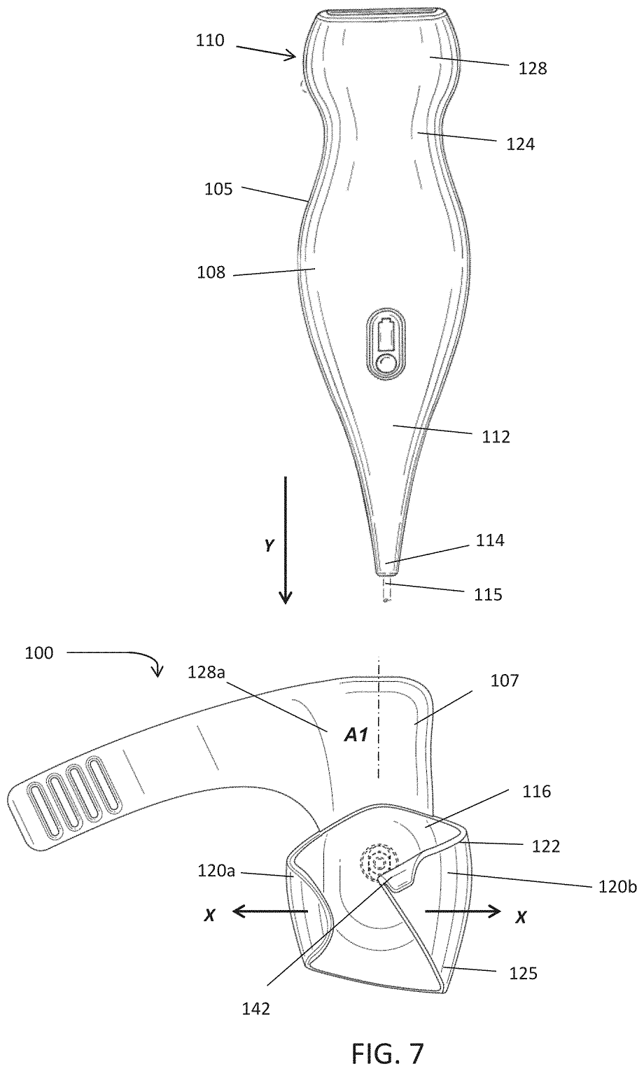

In some embodiments, the probe may be wider at a central region of the probe (see, e.g., FIG. 7, showing a probe 110 that tapers inwardly above and below the central region 108 of the probe). In such embodiments, the cradle may extend at least partly above the central region of the probe when the probe is seated in the cradle. The cradle also may extend at least partly below the central region of the probe when the probe is seated in the cradle.

In some embodiments, the cradle may be arranged to have a snap-on engagement with the probe. For example, in some embodiments, the cradle may be arranged to deflect outwardly as the probe is being seated in the cradle and to return back to the non-deflected position once the probe is in the cradle. As will be appreciated, in such an example, the cradle (such as arms of the cradle) may be biased toward the non-deflected position such that the cradle returns to the non-deflected position when the probe is seated in the cradle. In some embodiments, the resiliency of the material forming the cradle may provide some or all of the biasing toward the non-deflected position. The cradle does not necessarily have to return all the way to its non-deflected position when holding the probe.

In some embodiments, the cradle may have a shape and size that corresponds to the shape and size of at least a portion of the probe (e.g., a central region of the probe body). In such embodiments, when the probe is inserted downwardly (e.g., vertically) into the cradle, a portion of the probe body, such as a tapered portion below the central region, may contact the cradle and cause the cradle to deflect outwardly and open the cradle until the central region of the probe may be seated in the cradle. For example, the cradle may include first and second arms that may move outwardly and away from one another when the probe is inserted into the cradle. Once the probe is in the seated position, the arms may move toward their original, non-deflected positions around the probe. In some embodiments, the arms may move all the way to their original, non-deflected positions.

In some embodiments, the strap may be attachable to the cradle to secure the probe in the holster. For example, in some embodiments, the strap includes one or more openings into which a corresponding hook on the cradle may be inserted. In some embodiments, the strap may be arranged for a quick release from the cradle. For example, at least a portion of the hook may be angled such that the opening in the strap may be quickly moved off of the hook, as will be described.

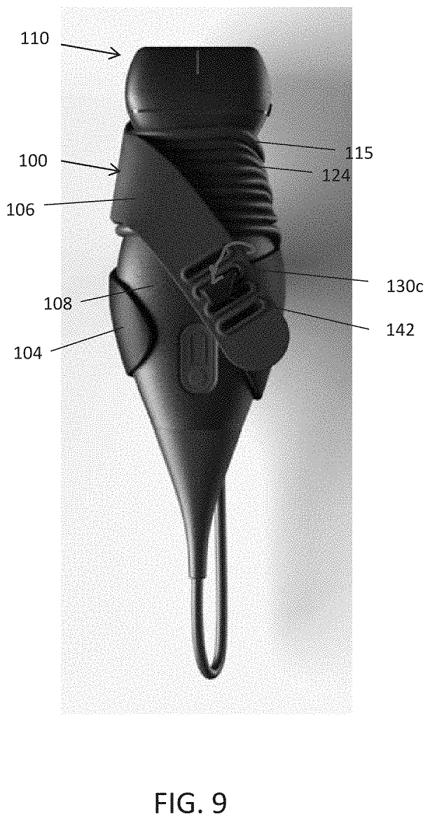

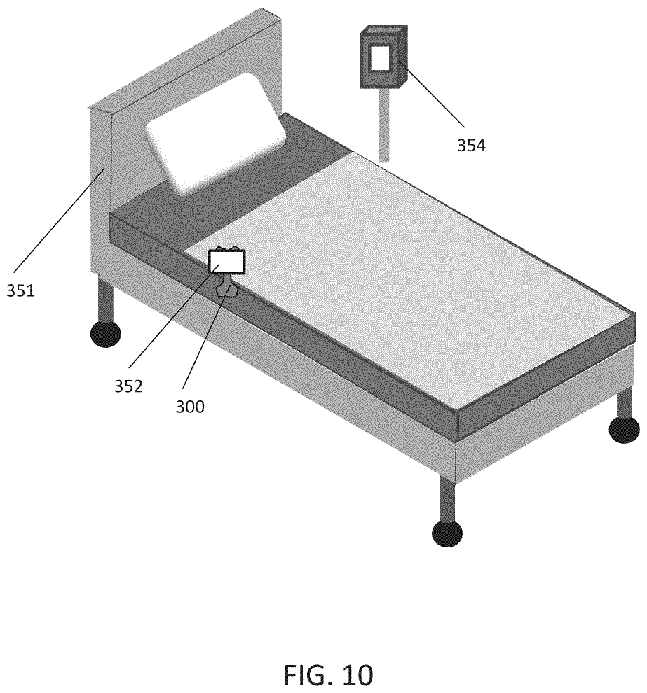

In some embodiments, the strap is arranged to extend diagonally across at least a portion of the probe. For purposes herein, extending diagonally may mean that the strap extends at an angle relative to a longitudinal axis of the probe. In some embodiments, the strap may extend over the shoulder of the probe, across a front of the probe, and be secured to the cradle of the holster. In some embodiments, the strap may extend diagonally to, or in some embodiments diagonally over, the central region of the probe. In some embodiments, the diagonal extension of the cable relative to the longitudinal axis of the probe body may maintain the cord on the neck of the probe (see, e.g., FIG. 9). The diagonal extension of the strap also may be arranged to maintain the position of the probe relative to the holster. For example, the strap may maintain the probe in a vertical position (e.g., with the head of the probe pointing upwardly) when the probe is secured in the holster.

In some embodiments, the strap may include a flexible material. For example, in some embodiments, the strap may be formed of a silicone material, although the strap may be formed of other suitable materials. In some embodiments, the strap may be stretched over the probe to tightly secure the probe to the holster. In such embodiments, the tensioned securement of the probe via the stretched strap may maintain the cord on the probe and may maintain the relative position of the probe and the holster.

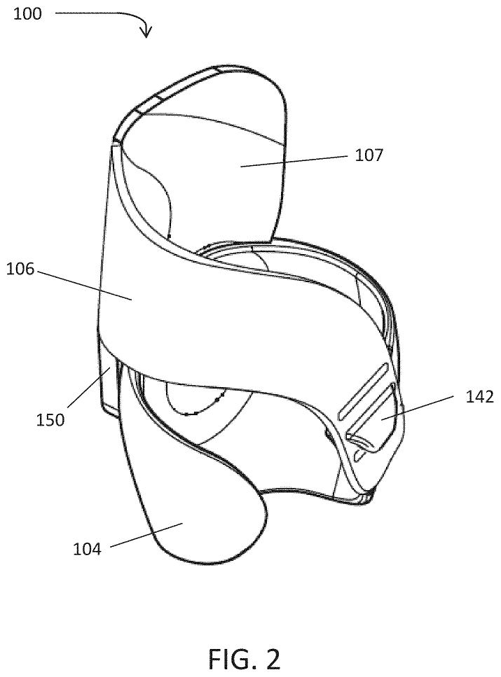

In some embodiments, the holster may be arranged to at least partially collapse when the probe is removed from the holster. For purposes herein, being at least partially collapsible means that at least a portion of the holster does not maintain a position or arrangement held by the portion of the holster when the probe is not in the holder (see, for example FIG. 2 showing the securement arrangement of the holster). For example, the holster may at least partially collapse by having a first portion of the holster fold or flop over onto a second portion of the holster.

In some embodiments, the holster may be formed of both rigid and flexible materials. For example, the cradle may be formed of a rigid material, such as polycarbonate plastic, while the strap is formed of the above-noted flexible material. In such an example, the cradle may be formed of a material that is more rigid than the flexible material used to form the strap In some embodiments, the cradle is arranged to have a thickness such that the cradle (e.g., the arms) may deflect when the ultrasound probe is being seated in the cradle. In some embodiments, the cradle may include high impact strength. In some embodiments, the holster also may include a support member arranged to support the probe, as will be described. In some embodiments, the support member also may be formed of a flexible material (e.g., silicone). In some embodiments, the strap and/or support member may be collapsible into, onto, or around the cradle when the probe is removed from the holster. In some embodiments, the rigid cradle may be arranged to have a smaller profile as compared to other rigid holsters such that the when the probe is in use (e.g., for testing) the rigid cradle may be less obstructive to the clinician wearing the holster.

In some embodiments, the holster may be arranged to hold the probe relative to the user in numerous orientations. For example, in some embodiments, the holster may include a clip that is moveable, such as rotatable, relative to the holster. In some embodiments, the user may move the clip relative to the holster, or move the holster relative to the clip, such that when the clip is attached to the user's clothing, the probe is aligned in a desired position relative to the user. For example, the user may attach the clip to his or her clothing such that a head of the probe is facing upwardly, to the side, or in another suitable direction. The holster also may include a clip that is removable (e.g., removable from the support member, cradle, and/or strap). In some embodiments, the user may leave the clip attached to the user's clothing or other personal item. In such embodiments, the user may then orient the probe in the desired arrangement, the probe being secured in the holster (e.g., via the cradle and strap), and the then attach the holster to the clip on the user's clothing, such as via a coupler that engages with the clip.

Although the holster is described as being worn by the clinician while the probe is being used for testing, it will be appreciated that the user may also remove the holster when the probe is in use. For example, the user may remove the holster (including the clip) from user's clothing.

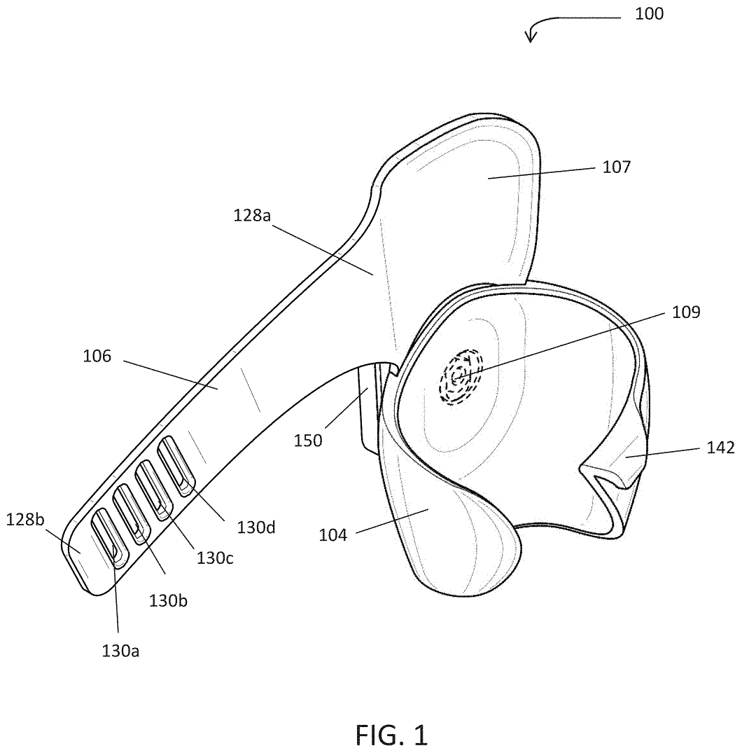

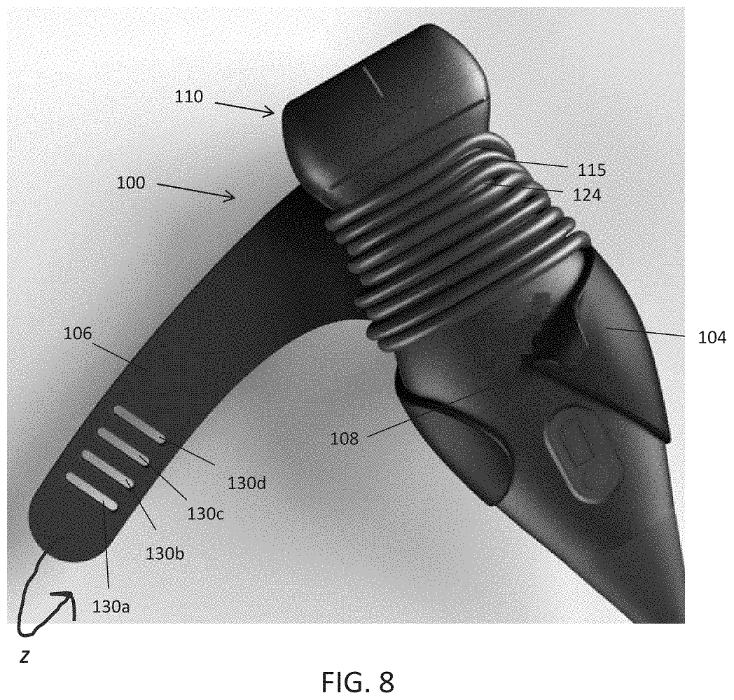

FIGS. 1 and 2 show a holster 100 for retaining an ultrasound imaging device, such as an ultrasound probe, on a user according to embodiments of the present disclosure. As shown in these views, the holster 100 may include one or more retainers for retaining the ultrasound probe in the holster. For example, the holster may include a cradle 104 arranged to hold the ultrasound probe in the holster and a strap arranged to secure the probe to the holster. In some embodiments, the holster 100 also may include an support member 107 arranged to support at least a portion of the probe. For example, as shown in FIG. 8, the support member may include an elongate member positioned behind at least a neck of the ultrasound probe when the probe is seated in the cradle.

Although the holster is shown as having only two retainers (e.g., a strap and a cradle) in these views, in some embodiments, the holster may include one, two, three, four, or more retainers for holding the probe in the holster. For example, the holster may include only a cradle to hold the ultrasound probe in some embodiments or may include a cradle and two straps.

In some embodiments, as shown in FIG. 1, the support member 107 may be fixedly attached to the strap. For example, the support member may be integrally formed with the strap in some embodiments. In such an example, the support member and strap may be formed of the same flexible material. As will be appreciated, the strap also may be attachable to the support member. In some embodiments, as also shown in FIG. 1, the cradle may be attached to the support member. In some embodiments, the cradle may be attached to the support member via a fastener 109. As will be appreciated, the cradle may be attached to the support member via any suitable arrangement.

In some embodiments, the cradle 104 may be arranged to hold at least a portion of the probe body 105 (see FIG. 7-9) of the ultrasound probe 110 when the ultrasound probe is inserted into the holster. For example, in some embodiments, as shown in FIGS. 8 and 9, the cradle may be arranged to hold a central region of the probe. In such embodiments, the shape of the cradle may correspond to the shape of the selected portion (e.g., central region) of the body of the ultrasound probe. The shape and size of the cradle also may correspond to other portions of the body in other embodiments.

As shown in FIG. 7, for example, a first portion 112, such as a base or handle, of the probe body 105 may be tapered, with a diameter of the probe decreasing toward a first end 114 of the probe. In some embodiments, a cable 115 may be attached at the first end 114 of the probe. The handle may taper from the first end toward a central region 108 of the probe. In some embodiments, the probe body also may taper from the central region of the probe toward the neck 124 of the probe.

In some embodiments, as shown in FIGS. 1, 4, and 7, the cradle 104 also may have a body 116 with a corresponding taper. For example, as shown in FIG. 7, the diameter of the cradle may increase from a first end 122 toward a position in between the first and second ends of the cradle, and thereafter decrease toward the second end 125 of the cradle. As will be appreciated, the cradle may have other suitable shapes in other embodiments for holding the ultrasound probe in the holster.

In some embodiments, as shown in FIG. 8, the cradle is arranged to wrap around at least a portion of the ultrasound probe to hold the ultrasound probe in the holster. As will be appreciated, the cradle need not extend around an entire circumference of the ultrasound probe. As shown in FIG. 1, for example, the cradle may include first and second arms 120a, 120b, each of which wraps around a portion of the probe. As will be further appreciated, the cradle may extend around an entire circumference of the probe in other embodiments.

As will be appreciated, the arms may be the same shape and size and encompass the same volume of probe body. The shape and size of the arms also may vary from arm to arm such that the arms encompass different volumes of the probe. As will be further appreciated, the arms may have any suitable shape and size. For example, the arms may be substantially rectangular, circular, square, triangular, oval, other polygonal shape, or other suitable shape. In some embodiments, the cradle may include two arms, as is shown, although the cradle may include more than two arms in other embodiments. The cradle also may be a single unitary piece in other embodiments.

In some embodiments, the cradle may hold the probe in an over-center arrangement. For example, the arms may be flexibly resilient and resist insertion of the probe into the cradle. In some embodiments, once the widest point of the probe body passes the upper edge of the arms while being inserted, the arms may no longer resist the insertion, and instead may start to push the probe to its cradled position, or at least no longer resist the cradling of the probe.

In some embodiments, as shown in FIG. 8, when the probe is fully seated in the cradle, the cradle may extend above central region of the probe. The cradle also may extend below the central region of the probe body. For purpose herein, the central region may mean a region of the probe in between the head of the probe and the first end 114 of the probe. For example, the central region may be a region equidistant between the head of the probe and the first end of the probe. In some embodiments, the central region of the probe is the widest region of the probe. For example, the diameter of the probe may be largest at the central region of the probe.

In some embodiments, each of the arms 120a, 120b of the cradle may extend to the same height above and/or below the central region of the probe. The arms also may extend to different heights above and/or below the central region of the probe (see, e.g., FIG. 7). For example, in some embodiments, only one of the arms may extend above the central region of the probe.

In some embodiments, as shown in FIG. 8, the first and second arms 120a, 120b wrap around a front portion of the probe. In such embodiments, at least a portion of the arms supporting the front of the probe may extend above and/or below the central region of the probe. In some embodiments, only one of the arms may have a front supporting portion that extends above and/or below the central region of the probe.

In some embodiments, as shown in FIG. 7, the ultrasound probe 110 may be slid in a direction (see arrow Y) into the cradle. In such embodiments, the direction Y is substantially parallel to a longitudinal axis A1 of the holster As will be appreciated, the ultrasound probe also may be slid into the cradle at other suitable angles, such as angled relative to the holder.

In some embodiments, as the probe is slid into the cradle, the first and second arms 120a, 120b move away from one another and/or deflect in a direction outwardly from the longitudinal axis of the cradle (see the arrows labeled X in FIG. 7) to allow the probe to be moved into the cradle. In some embodiments, a downward-facing surface of the probe, such as the downward-facing tapered surface between the central region 108 and the first end 114 of the probe, may contact the arms to cause the arms to move away from one another. In some embodiments, once the central region of the probe passes the first end 122 of the cradle and the probe is seated in the cradle, the first and second arms 120a, 120b may move back toward one another, in some cases to their original non-deflected position, to hold the probe in the holster. In some embodiments, the arms remain slightly deflected even after the probe has been fully cradled, which may help restrain the probe from movement within the cradle.

In some embodiments, the ultrasound probe also may be maintained in the cradle via the corresponding shapes of the ultrasound probe and the cradle. For example, as shown in FIG. 7, as both the cradle and the ultrasound probe are tapered, the diameter of the second end 125 of the cradle 104 may be smaller than a diameter of the ultrasound probe above the first end 114 of the ultrasound probe (e.g., toward a central region of the probe). In such an example, when the probe is inserted into the cradle, the probe may not slide out of the opening at the first end 122 of the cradle. As will be appreciated, in such an example, the force of gravity acting on the probe may act to urge the ultrasound probe toward the second end 125 of the cradle when the probe is inserted in the cradle.

In some embodiments, as shown in FIGS. 8 and 9, a portion of the probe may extend outwardly beyond the cradle when the ultrasound probe is held in the cradle. For example, the first end (e.g., the base of the probe) and the cable may extend outwardly beyond the first end 112 of the cradle.

In some embodiment the end of the cord may include a clip, snap, or other faster which may be attached to another portion of the cord (e.g., to hold the cord being wrapped around the fingers and/or probe). As will be appreciated, the clip may include a channel into which the portion of the cord may be inserted. In such embodiments, the shape of the channel may correspond to the shape of the cord.

In some embodiments, as shown in FIGS. 1, 2, 4, 6, and 7-9, the holster may include a strap 106 configured to secure the probe to the holster. In some embodiments, such as that shown in FIG. 9, the strap may extend around only a portion of the probe, such as the neck of the probe and the front of the probe, extending toward the central region. In some embodiments, the strap may be stretched to secure a cord to the probe and also to the holster.

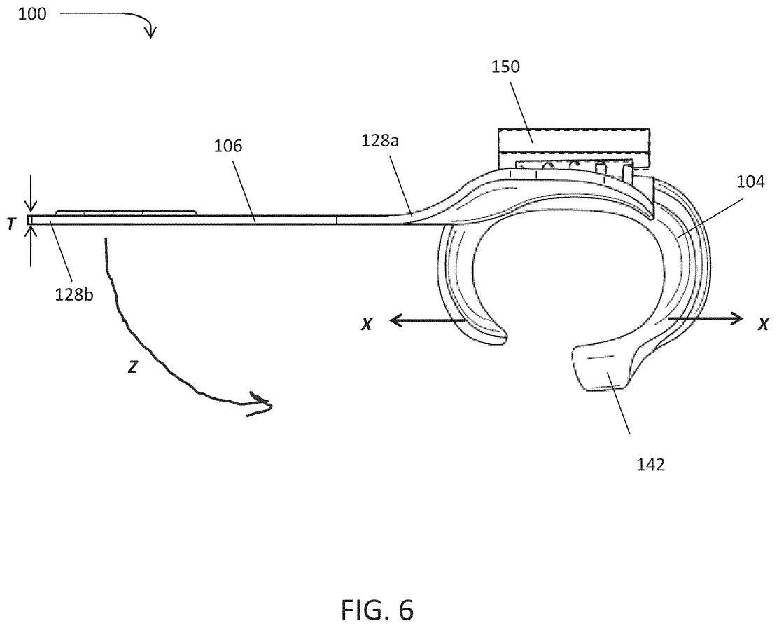

As shown in FIGS. 4 and 7, in some embodiments, a first end 128a of the strap is attachable to the support member 107. For example, the strap may be integrally formed with the support member. In some embodiments, the strap includes one or more openings 130a-d at the second end 128b of the strap for securing the strap to the cradle 104. For example, the user may stretch the strap over the probe (see the arrows labeled Z in FIGS. 6 and 8) and may insert a hook 142 of the cradle into one of the corresponding openings 130a-d. As will be appreciated, the user may insert the hook into the opening that allows the user to tightly stretch the strap across the probe body to create a tensioned securement. See, for example, FIG. 9, which shows the hook 142 inserted into third opening 130c from the second end 128b of the strap.

As shown in FIGS. 4 and 7, the strap may extend outwardly from the a first lateral side of the support member and at an angle relative to the longitudinal axis of the holster A1. In some embodiments, to attach the strap to the cradle, the strap is wrapped around the first side of the probe, crossing over a centerline of the probe, the centerline being parallel with the longitudinal axis of the probe, to attach the opening to the hook 142 on the second arm of the cradle. As will be appreciated, the second arm is located on a side of the cradle adjacent to the second lateral side of the support member, which is opposite to the side to which the strap is attached.

As also shown in FIG. 4, in some embodiments, the width of the strap decreases from the first end 128a of the strap toward the second end 128b of the strap. For example, in some embodiments, a width W1 at the first end of the strap may be between about 30 mm and about 34 mm, or between about 32 mm and about 33 mm. In such embodiments, the width of the second end of the strap 129b may be between about 21 mm and about 25 mm, or between about 23 mm and about 24 mm. As will be appreciated, the width of the strap may decrease in any suitable fashion. For example, the width of the stray may decrease in a tapered fashion between the first and second ends. The also may decrease in a stepped fashion. For example, a first portion of the strap may have a first width, while a second portion of the strap may have a second width smaller than the first.

In some embodiments, the width of the first end 128b of the strap may correspond to the size of the neck of the probe. For example, as shown in FIG. 9, the strap is arranged to extend over an entire first side, such as the left side, of the neck of the probe. As will be appreciated, the width of the first end of the strap also may correspond to the region of the probe around which the cord is wrapped. In this regard, the width of the first end may correspond to the size of the wrapped cord around the neck.

In some embodiments, as shown in FIG. 6, the strap may have a thickness T of between about 1 mm and about 3 mm. For example, the strap may be about 2 mm thick in some embodiments. In embodiments in which the strap is integrally formed with the support member 107, the strap and support may have the same thickness, such as about 2 mm. As will be appreciated, the support member 107 may be thicker than the strap in some embodiments.

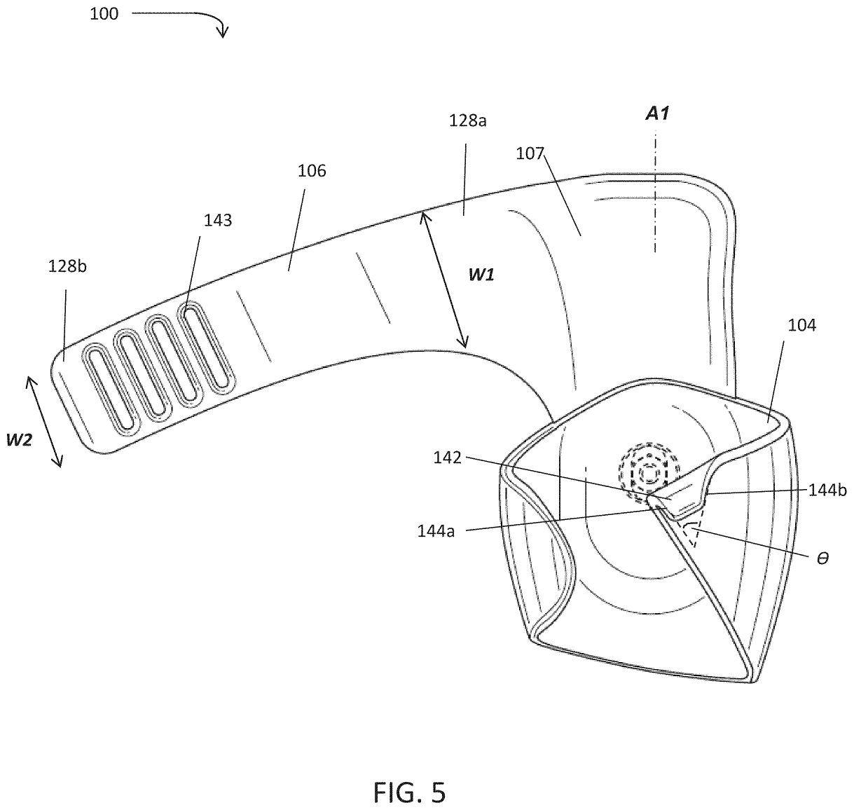

In some embodiments, as shown in FIGS. 4 and 5, each of the openings on the strap may include a raised section 143 that extends around a periphery of the opening. In some embodiments, the raised section 143 is arranged to minimize tearing of the strap as the openings are repeatedly engaged and disengaged with the hook. In some embodiments, the raised section may be between about 0.5 mm and about 1.5 mm in thickness. For example, the raised section may be about 1 mm thick. As will be appreciated, this raised thickness extends on top of the strap, which as noted above, may be about may be about 2 mm thick.

In some embodiments, the holster is arranged for quick release of the probe. For example, in some embodiments, the holster is arranged such that the strap may be quickly released from the hook on the cradle. In some embodiments, as shown in FIG. 5, the hook 142 includes first and second sides 144a, 144b. The hook also includes a channel for receiving at least a portion of the strap when the hook is inserted in one of the openings 130a-d. As shown in FIG. 5, the first and second sides 144a, 144b of the hook do not extend parallel to one another. Instead, the second side 144b is angled relative to the first side (see the angle labeled θ in FIG. 5). In some embodiments, the angled second side 144b of the hook is arranged to allow the opening to be quickly slid over the second side of the hook when the user pulls on the strap (see the arrow (see the arrow in FIG. 9).

As shown in FIGS. 8 and 9, in some embodiments, the support member may be arranged to support a rear portion of the probe. In some embodiments, the support member may include one or more ribs 156, which, in some embodiments, are arranged to provide structural stability and/or rigidity to the flexible support member. For example, ribs may be arranged to stop the flexible support member from flopping over, such as when the support member is positioned behind the probe. In some embodiments, the ribs may help the support member to remain behind the probe when the strap is stretched to create the tensioned securement around the probe. In such embodiments, the support member and strap may be wrapped all the way around the probe, creating the tensioned securement of the probe on the holster.

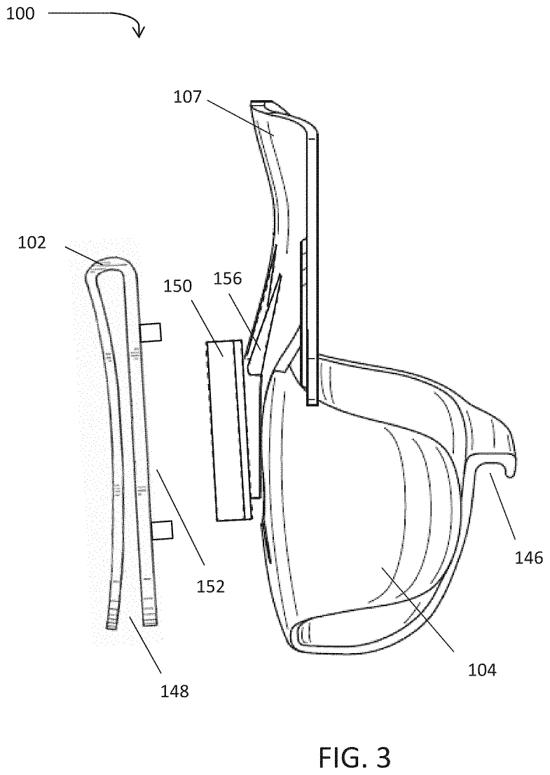

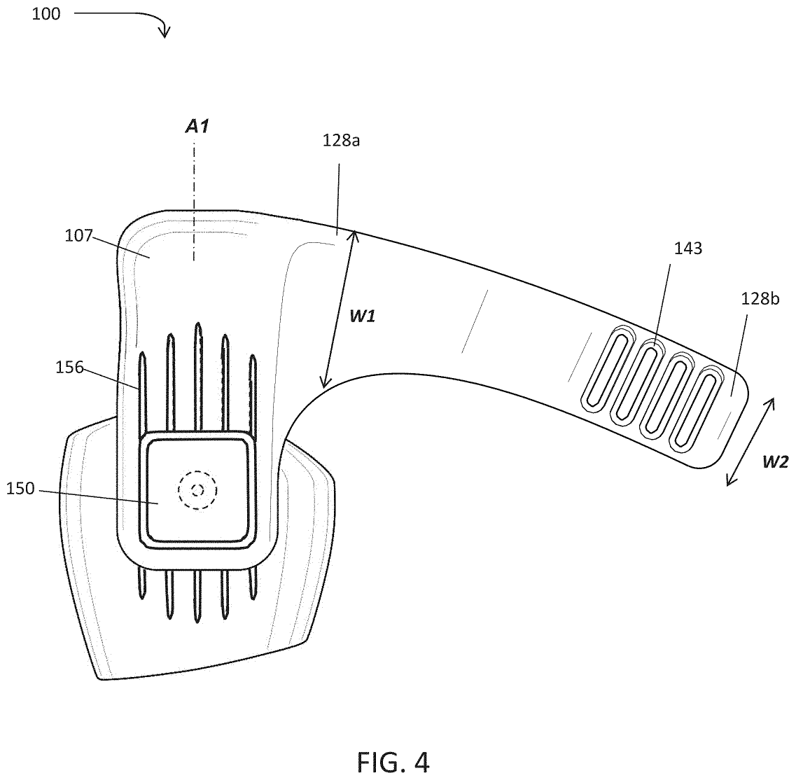

In some embodiments, as shown in FIGS. 3-5, the ribs extend outwardly from a rear side of the support member. In some embodiments, the ribs 156 extend around at least a portion of a clip of the holster. For example, the ribs may extend around at least a portion of a coupler 150 that engages with a clip 102 (see FIG. 3) that may be attached to user's clothing and/or personal item. In some embodiments, the ribs are between about 1 mm and 2.5 mm wide, more specifically about 2 mm±0.5 mm wide. In some embodiments, the ribs may be between about 2 mm and about 3 mm thick. As will be appreciated, the width and thickness of the ribs may be the same, although they may vary from rib to rib. The ribs may have any suitable cross-sectional shape (e.g., rectangular, oval, triangular).

In some embodiments, the holster may include the clip 102 to attach the holster to a portion of the user's clothing, such as to the waist area of a pair of pants, to a skirt, or to a pocket of a coat. In some embodiments, as shown in FIG. 3, the clip 102 may include an opening 148 into which a piece of the user's clothing may be inserted. As will be appreciated, the opening 148 may have any suitable shape and size as this aspect of the disclosure is not limited in this regard. In some embodiments, the clip may include an actuator that may be pressed by the user to access the opening 148 for insertion of a portion of the user's clothing. In such embodiments, the actuator may be biased toward a closed position such that when the user releases the actuator, the opening may close to secure the clip to the user's clothing. As shown in FIG. 3, the opening also may remain open all the time, such that the user may simply slide the clip onto the user's clothing or other personal item.

In some embodiments, the clip may be rotatably attached to the holster. For example, the clip may be rotatably attached to the rear of the support member 107. In such embodiments, the holster may be rotated to adjust the position of the holster and probe, or the position of an empty holster, relative to the user when the holster is attached to the user's clothing. For example, the user may rotate the holster out of the way when the user is using the probe for imaging and/or treatment.

In some embodiments, as shown in FIG. 3, the clip may be removable attachable to the holster, such as to the rear surface of the cradle and/or to the rear surface of the support member. In such embodiments, the holster may include the coupler 150, which is attachable to the clip. For example, as shown in FIG. 3, the clip may have a recess 152 into which the coupler is inserted (e.g., snapped onto). During use, the user may secure the probe in the holster and thereafter attach the holster to the clip (e.g., via the coupler) such that the probe is oriented in the desired alignment. In some embodiments, the clip includes an actuator that the user may press o disengage the coupler from the clip. In some embodiments, the actuator may be spring loaded.

Although the holster is described as being removably attachable to a clip on a piece of clothing or other personal item, the holster also may be attachable to (e.g., snapped onto) other items in a hospital, clinic, or other medical site, such as an ambulance or a military environment. For example, a clip or other connector having a recess 152 that corresponds to the coupler 150 on the holster may be attached to a patient's hospital bed, an intravenous pole, or another piece of medical equipment or fixture. In such an example, the clinician may remove the holster from the clip on his or her belt, and then attach the holster to the clip or connector on the medical equipment and/or fixture via the coupler. In some embodiments, this modularity may allow the clinician to keep the ultrasound imaging device within arm's reach while working on a patient.

Although a clip is shown and described for allowing the holster to be held on the user, other arrangements may be used to attach the probe to the user's clothing or to otherwise allow the user to wear the holster. For example, the holster may include a lanyard or other necklace that the user may place around his or her neck to wear the holster. The holster also may be attached to a belt or strap, that the user may secure around his/her waist. As will be further appreciated, the holster need not have a clip. For example, the holster may include only retainers, as will be described, for retaining the ultrasound probe in the holster and protecting the ultrasound probe. In such an example, the user may simply place the holster, with an attached ultrasound probe, in a pocket of his/her jacket or in his/her bag.

The ultrasound imaging device 110 may be a wired or wireless imaging device. In embodiments in which the ultrasound imaging device 110 is wired, the holster 100 may include an opening, extension, or other configuration for allowing the wire/cable to fit into the holster and/or to wrap around the holster. For example, as shown in FIG. 1, in some embodiments, the holster may include a slit between the first and second arms 120a, 120b through which the cable 115 of the probe may extend.

Although the holster is shown as having a cradle and strap for holding the ultrasound probe relative to the holster, other suitable retaining mechanisms may be used to hold the probe in the holster. For example, in some embodiments, the probe may include a fastener that engages with a corresponding fastener on the holster. The probe also may include a protrusion that is received in a corresponding opening on the holster. The probe may further include a snap, clip, or hook that engages with a corresponding snap, clip, or loop on the holster. As will be appreciated, the holster and probe also may have other suitable arrangements in other embodiments.

In some embodiments, as shown in FIG. 10, the holster may include one or more retainers arranged to hold a portable electronic device, such as a smart phone, when the user is performing imaging and/or treatment on a patient. In some embodiments, the fingers used to hold the probe in the holster may be used to hold the portable electronic device. In other embodiments, the holster may have other suitable retainers (e.g., a second set of fingers) to hold the portable electronic device. As shown in FIG. 10, the clip (not visible) of the holster may be used to attach the holster to a patient's bed 351, such as by clipping the holster onto a blanket or sheet. The portable electronic device 352, in turn, may be attached to the holster 300 for viewing by the user. As will be appreciated, the holster may be attached to other suitable surfaces, such as to a pole of an intravenous dispensing machine. As will be further appreciated, the holster also may include a handle that the user may grasp to view an attached portable electronic device while performing imaging and/or treatment on a patient.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. A holster for holding an ultrasound imaging device on a user, the holster comprising;
a cradle arranged to receive at least a portion of the ultrasound imaging device; and
a strap having first and second ends, wherein the first end of the strap is attached to the holster and the second end of the strap is removably attachable to the cradle to secure the ultrasound imaging device in the cradle.

2. The holster of claim 1, further comprising a support member arranged to support at least a neck of the ultrasound imaging device when the ultrasound imaging device is seated in the cradle, wherein the first end of the strap is attached to the support member.

3. The holster of claim 1, wherein the cradle includes first and second arms.

4. The holster of claim 3, wherein the first and second arms are arranged to move outwardly and away from one another when the ultrasound imaging device is inserted into the cradle.

5. The holster of claim 3, the first and second arms deflect outwardly away from one another when the ultrasound imaging device is inserted into the cradle.

6. The holster of claim 3, wherein, when the ultrasound imaging device is seated in the cradle, at least a portion of the cradle extends above a central region of the ultrasound imaging device.

7. The holster of claim 6, wherein a diameter of the ultrasound imaging device is largest at the central region of the ultrasound imaging device.

8. The holster of claim 1, wherein the cradle is configured to wrap around at least a portion of the ultrasound imaging device.

9. The holster of claim 1, wherein, when the ultrasound imaging device is secured in the holster, and the first end of the strap is attached to the cradle, the strap extends diagonally across a front portion of the ultrasound imaging device.

10. The holster of claim 1, wherein the strap is arranged to extend around a least a portion of a neck of the ultrasound imaging device.

11. The holster of claim 1, further comprising a clip arranged to attach the holster to a user's clothing or personal item.

12. (canceled)

13. (canceled)

14. The holster of claim 2, wherein the strap extends outwardly from a first lateral side of the support member.

15. The holster of claim 14, wherein the strap is attachable to a second arm of the cradle, the second arm of the cradle being adjacent to a second lateral side of the support member which is opposite to the first lateral side.

16. The holster of claim 1, wherein a width of the first end of the strap is larger than a width of the second end of the strap.

17. The holster of claim 1, wherein the cradle comprises a hook, and wherein the second end of the strap includes one or more openings for engaging with the hook on the cradle.

18. The holster of claim 4, wherein the first and second arms are arranged to move toward a non-deflected position as the ultrasound imaging device is seated in the cradle.

19. The holster of claim 2, wherein the strap is integrally formed with the support member.

20. The holster of claim 2, wherein the support member includes one or more outwardly extending support ridges.

21. The holster of claim 1, wherein the strap is formed of a flexible material and the cradle is formed of a material which is more rigid than the flexible material of the strap.

22. (canceled)

23. A method comprising:
inserting an ultrasound imaging device into a cradle of a holster;
stretching a strap over at least a portion of the ultrasound imaging device; and
securing the strap to the cradle.

24-29. (canceled)

30. A holster for holding an ultrasound imaging device on a user, the holster comprising;
a cradle arranged to receive at least a portion of the ultrasound imaging device, the cradle having first and second arms arranged to wrap around at least a portion of the ultrasound imaging device; and
a strap having first and second ends, the first end of the strap being attached to the holster;
wherein the first and second arms are arranged to deflect outwardly away from one another when the ultrasound imaging device is inserted into the cradle.

31-35. (canceled)
